(19) United States
(12) Patent Application Publication
(10) Pub. No.: US 2024/0299613 A1
(43) Pub. Date: Sep. 12, 2024

(54) ABSORBENT MATERIAL FOR WOUND DRESSINGS AND METHODS OF MAKING THE SAME

(71) Applicant: DelStar Technologies, Inc., Alpharetta, GA (US)

(72) Inventors: Andrew G. Platt, Middletown, DE (US); John Cox, Alpharetta, GA (US); Yasar Kiyak, Alpharetta, GA (US); Lenny Pompeo, Alpharetta, GA (US); Andrew Scarff, Alpharetta, GA (US)

(21) Appl. No.: 18/595,506

(22) Filed: Mar. 5, 2024

Related U.S. Application Data
(60) Provisional application No. 63/489,161, filed on Mar. 8, 2023.

(51) Int. Cl. A61L 15/42; A61F 13/0206; A61L 15/22

(52) U.S. Cl. CPC A61L 15/425; A61F 13/0206; A61L 15/225

(57) ABSTRACT

Provided herein are absorbent materials for wound dressings, a bandage comprising an absorbent pad and methods for making the absorbent pad. The absorbent pad comprises a first layer comprising a polymer, such as an extruded apertured polymeric film, and a second layer thermally bonded to the first layer. The second layer comprises at least one thermally bondable fiber, such as a bicomponent fiber having at least a first material with a higher melting point than a second material. The first and second layers are bonded to each other such that the three-dimensional structure and the inherent porosity of the polymeric mesh is substantially maintained, resulting in a bilayer absorbent pad with increased absorbency and enhanced wicking capabilities.

### CROSS-REFERENCE TO RELATED APPLICATIONS

This application claims the benefit of U.S. Provisional Application Ser. No. 63/489,161, filed Mar. 8, 2023, the complete disclosure of which is incorporated herein by reference for all purposes.

### TECHNICAL FIELD

The present disclosure generally relates to wound dressings, such as bandages, and more particularly to absorbent materials for wound dressings and methods for manufacturing the same.

### BACKGROUND

Absorbent materials are used in wound care to protectively cover the wound site, absorb moisture and exudate, and facilitate healing. Such materials typically comprise nonwoven fabrics that include at least one fluid pervious layer for contact with the surface of the healing wound. Traditionally, nonwoven fabrics that make up absorbent pads used in bandages contain thermoplastic polymeric fibers o confer strength to the pads. The material can be fibrous, porous, natural, or synthetic. Preferred fibers are synthetic resin fibers, particularly olefinic fibers, such as polyethylene, polypropylene, or a blend, and the absorbent article may incorporate other material such as polyester, rayon, cotton, or blends thereof. Polyester resins such as polyethylene terephthalate, polyethylene naphthalate, polylactic acid, and others, have been used in the art.

One of the major goals in the art has been to improve the absorbency and wicking capability of absorbent pads, while maintaining a relatively thin material that is cost-effective and comfortable for use. Determining the optimal fiber materials, arrangement of the fibers, and configuration of the layers of these fibers can be challenging because certain aspects of the manufacturing process currently used in the field reduce the efficiency of the absorbent article. For example, configuration of the layers and choice of raw material may cause weak bonding to occur that compromises the absorbent article's structural integrity. This leads to poor absorbency and delamination of the fabric.

In one such conventional absorbent pad, a layer of nonwoven polymeric mesh is bonded to a layer of polyethylene terephthalate (PET) fibers. To ensure proper bonding of the polymeric materials, the fiber layers are commonly subjected to heat- and pressure-rolling using calendar rollers in a process called calendaring, that compresses and welds the layers together at high speed. The heat and high pressure are applied at the calendaring points of calendar rolls through which the fabric is applied.

Though very useful for fusing polymers, the continuous mechanical impact of high-pressure calendaring on the absorbent material may result in damage and collapse of the article's structure, such as, for example, the three-dimensional structure of the nonwoven polymeric mesh. The forces of calendar rollers may also disrupt the inherent porosity of the porous polymeric meshes. These pores are formed by the spaces within the three-dimensional arrangement of the fibers as well as openings, or apertures, formed therein. Consequently, these pores may become misshaped during the pressing process. This is especially counterproductive because the apertures are uniformly shaped to give the article a consistent pattern, each aperture shaped and spaced apart in a distance customized for the application. For example, uniformity of the geometry of the apertures within an absorbent pad in a bandage ensures that no “dead spots” are present from one layer to the next during usage, so that irregularly structured regions of the absorbent article do not impede fluid flow and prevent certain areas of the wound from breathing. Absorbent pads with a collapsed structure or irregularities within the structure present obstructed channels of flow for water, blood, sweat and discharge within the film. Moreover, irregularly spaced apertures do not allow water vapor to escape equally and evenly across the wound and thus, can increase the risk of wound maceration.

High-pressure calendaring changes the properties of the absorbent article, the nature of the apertures, and the wicking pattern, and thus, reduces the article's overall absorbency. Therefore, it would be desirable to provide improved absorbent materials for wound dressings that include thin polymer layers that may be bonded together without disrupting the three-dimensional structure of the material or impairing the porosity of the article, thereby improving the overall absorbency of the wound dressing.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

Absorbent pads for wound dressings and methods for making such absorbent pads are provided herein. The absorbent pads comprise a highly absorbent polymer composition, engineered using fibers and polymers that are arranged and bonded together without substantially disrupting the three-dimensional structure or the porosity of the material. The absorbent pads may, for example, be used as the layer in a bandage contacting the wound. The bandage may also comprise at least one other layer, such as an adhesive layer. Alternatively, the absorbent article may be used as a wrap-dressing itself. The absorbent composition is used to treat wounds such as, but not limited to, scrapes, cuts, punctures, and burns.

In one aspect, an absorbent pad for a wound dressing comprises a first polymer layer and a second layer thermally bonded to the first layer and comprising at least one thermally bondable fiber. The selection of polymeric fibers and their arrangements in a bilayer as disclosed herein are advantageous in that the composite pad is lightweight, and absorbent. In addition, the ability to thermally bond the first and second layers to each other eliminates the step of high-pressure calendaring in the manufacturing of the absorbent pad. Therefore, the three-dimensional structure and the inherent porosity of the absorbent pad is substantially maintained, resulting in a bilayer absorbent pad with increased absorbency and enhanced wicking capabilities.

In embodiments, the thermally bondable fiber comprises a biocomponent fiber having at least two different materials. The bicomponent fiber may be continuous (e.g., high loft) or discontinuous. The biocomponent fiber may comprise any suitable configuration, such as core/sheath with a concentric or eccentric core, side by side, segmented pie, island in the sea, hollow bicomponent fiber. hollow segmented pie, trilobal bicomponent fiber, mixed fibers, striped fibers, conductive fibers and the like. The bicomponent fiber may have a solid or a hollow core.

In embodiments, the bicomponent fiber comprises first and second polymer materials. The second polymer material has a lower melting temperature than the first polymer material.

In an exemplary embodiment, the bicomponent fiber comprises a core and a sheath. The core may be concentric or eccentric with the sheath. The core comprises a first material and the sheath comprises a second material. The second material has a lower melting temperature than the first material. In an alternative embodiment, the first material has a lower melting temperature than the second material.

The core may comprise any suitable material, such as polyethylene terephthalate (PET), polylactic acid (PLA), polypropylene (PP) or combinations thereof. In an exemplary embodiment, the core comprises PET.

The sheath may comprise any suitable material, such as polyethylene (PE) high density polyethylene (HDPE), low-melting polyethylene terephthalate (CoPET), low-melting polylactic acid (PLA), polypropylene (PP) and combinations thereof. In an exemplary embodiment, the sheath comprises PE.

In one exemplary embodiment, the bicomponent fiber comprises a PET core surrounded by a PE sheath. The arrangement of the PET core and PE sheath provide an easily bondable composition using thermal bonding due to, for example, the differences in melting temperature of the two components. Additionally, the method of bonding the fiber layer to the first polymer layer adjacent to the fiber layer may employ types of bonding which do not include high-pressure mechanical forces. Ultimately, these materials and their configuration and processing obviate the need for bonding techniques that disrupt the three-dimensional structure of the composite and allow the article to perform with greater efficiency.

In embodiments, the ratio by weight of the core and the sheath is about 30/70 to about 70/30, preferably about 40/60 to about 60/40. This range maintains optimal overall density of the absorbent pad. Density is an important consideration of the pad, as values too high or too low may render the pad uncomfortable or weak and ineffective.

In some embodiments, a plurality of polyethylene sheaths are randomly configured in the second layer. Alternatively, the plurality of polyethylene sheaths may be configured to be adjacent to one another. In other embodiments, the plurality of sheaths may comprise a single layer, or multiple layers of sheaths. In some embodiments, they are configured in a structured pattern of intersecting sheaths.

In another embodiment, the bicomponent fiber has a configuration that may include side by side, segmented pic, island in the sea, hollow bicomponent fiber. hollow segmented pie, trilobal bicomponent fiber, mixed fibers, striped fibers, conductive fibers and the like. The bicomponent fiber may have a solid or a hollow core. The fiber includes at least first and second polymers having different melting temperatures and having ratio by weight of about 20/80 to about 80/20.

In embodiments, the second layer further comprises at least one non-thermally bondable fiber. The non-thermally bondable fiber has a melting point that is at least about 5° C., or at least 15° C. higher than the melting point of the thermally bondable fiber. In an exemplary embodiment, the non-thermally bondable fiber has a melting point higher than the melting point range (i.e., the “pasty range”) of the thermally bondable fiber such that the thermally bondable fiber completely melts before the non-thermally bondable fiber begins to melt.

The non-thermally bondable fiber may comprise any suitable material, such as polyethylene terephthalate (PET), polybutylene terephthalate (PBT) polylactic acid (PLA), polypropylene (PP) or combinations thereof. For example, when the sheath is selected as HDPE, the non-bondable fiber could be PET.

The non-bondable fiber may have any suitable cross-section, such as round, non-round, irregular, 4DG, trilobal, ribbon or the like. Non-round fiber cross-sections have more specific surface area which results in more liquid absorption. The non-thermally bondable fiber may comprise a bicomponent fiber.

In embodiments, the non-thermally bondable fiber comprises 50% or less by weight of the second layer, preferably less than 40% or less by weight of the second layer.

In some embodiments, the fibers in the second layer comprise a spin finish. The spin finish is selected from hydrophilic or hydrophobic FDA approved (for skin contact) spin finishes. Preferably, it is selected from hydrophilic spin finishes so that liquid absorption capacity is increased. However, it should be recognized that the spin finish may be a non-FDA approved spin finish as long as it does not harm the skin. The spin finish is content is no more than 2%.

In embodiments, the absorbent pad comprises a third polymer layer. The second layer may be positioned between the first and third layers. The second layer may also be thermally bonded to the third layer.

In embodiments, the first and/or third layers comprise a polymeric film or mesh. Suitable films or meshes include apertured films, perforated films, polymeric meshes, microporous films, netting and the like. The meshes or films may include, non-woven materials, woven materials, knitted materials and the like. In an exemplary embodiment, the polymeric film comprises an extruded film having apertures. The apertures may comprise pores or perforations and may have any suitable shape, such as hexagonal, square, diamond shaped. circular, oblong, triangular, rectangular or combinations thereof. Suitable materials for the extruded film include as polypropylene, polyethylene, high density polyethylene (HDPE) or combinations thereof.

In some embodiments, the absorbent pad has a thickness of about 40 mils to about 200 mils, preferably about 100 mils to about 130 mils. This range provides higher affinity bonding and reduces the amount of raw material required, thereby lowering higher manufacturing costs of the absorbent pad.

In certain embodiments, the absorbent pad has an absorbency between about 5 g/g to about 100 g/g/, or about 5 g/g/to about 40 g/g, or about 10 g/g to about 20 g/g.

The fibers can be manufactured by any suitable method, including, without limitation, meltblown, spunbond or spunlace, bicomponent spunbond, heat-bonded, carded, air-laid, wet-laid, extrusion, co-formed, needlepunched, stitched, hydraulically entangled or the like.

In one embodiment, the fibers are carded and thermally bonded to produce a web. The fibers have an absorbency of about 10 g/g to about 20 g/g or about 14 g/g to about 16 g/g and a basis weight of about 80 gsm to about 90 gsm.

In another embodiment, the fibers are spunbond by bonding together extruded spun filaments to create the web. The spunbond process may be more cost effective than other methods of producing the fibers and may create manufacturing efficiencies, such as higher throughput, lower maintenance, less space, increased automation, less scrap ratio, and a continuous manufacturing process (i.e., 24/7).

In certain embodiments, the spunbond fibers are continuous, or they may be formed directly from resin. In other embodiments, the spunbond fibers are staple fibers. The fibers may be naked prior to application of the coating (i.e., zero spin finish). The fibers may include a spin finish prior to the application of the coating. In certain embodiments, the staple fibers have a conventional spin finish of less than about 2%.

In an exemplary embodiment, the spunbond fibers are coated with a silicone-based coating that increases the absorbency of the pad. The coated spunbond fibers are then thermally bonded to the first polymer layer. The add-on weight of the silicone-based coating may be about 1.0 gsm to about 20 gsm, or about 1.5 gsm to about 15.5 gsm. In an exemplary embodiment, the coated spunbond fibers have an absorbency of about 5 g/g to about 20 g/g, or about 10 g/g to about 12 g/g. The coated spunbond fibers may have a lower basis weight than the carded fibers, or about 60 gsm to about 75 gsm.

In various embodiments, the silicone-based coating includes a silicone compound diluted in water or other suitable fluid such that the silicone compound comprises at least about two percent by weight of the coating, or at least about five percent by weight of the coating. In an exemplary embodiment, the silicone compound comprises about 10% by the weight of the coating.

In various embodiments, the silicone-based coating comprises a reactive silicone macroemulsion. The silicone emulsion may comprise, for example, dimethyl silicone emulsions, amino type silicone emulsions, organo-functional silicone emulsions, resin type silicone emulsions, film-forming silicone emulsions, or the like. In one embodiment, the reactive silicone macroemulsion comprises an amino functional polydimethylsiloxane and/or a polyethylene glycol monotridecyl ether. In an exemplary embodiment, the amino functional polydimethylsiloxane comprises about 30 to about 40 percent by weight of the coating. In embodiments, the polyethylene glycol monotridecyl ether comprises about 5 to about 10 percent by weight of the coating.

In some embodiments, the silicone-based coating further comprises an antistatic agent. The antistatic agent may comprise a cationic antistatic agent, an anionic antistatic agent, a quaternary antistatic agent, or a surfactant. The surfactant may comprise a non-rewetting thermodegradable surfactant/foaming agent.

In various embodiments, the silicone-based coating is applied by any suitable process including, but not limited to, spraying the fibers with the silicone-based coating, dipping the fibers in a vessel containing the silicone-based coasting, applying the silicone-based coating as a foam to the fibers, utilizing a metering rod or other leveling device to apply the coating, delivering the coating on the fibers with a coating head, such as a slot-die, or any combination of these techniques. The coating may be applied as a spin finish, or after a spin finish has been applied. The coating may be applied to naked fibers that have no spin finish. The coating may be applied to the staple fibers on which a typical spin finish is already applied.

In another aspect, a bandage is provided comprising an adhesive layer and an absorbent pad. The absorbent pad may be bonded to and extended across at least a portion of the adhesive layer. The absorbent pad comprises first and second layers. The first layer of the absorbent pad comprises a polymer and a second layer thermally bonded to the first layer and comprising at least one thermally bondable fiber.

This first layer may comprise a polymeric film as described above. The second layer may comprise any of the embodiments described above. The absorbent pad may include a third layer such that the second layer is positioned between the first and third layers. The bandage may also comprise other layers besides the adhesive and absorbent layers, such as, for example, padding layers, release layers, and others known in the art.

In another aspect, a method of making an absorbent pad for a wound dressing is provided. The method comprises providing a first polymer layer and a second fiber layer and thermally bonding or laminating the first layer to the second layer.

In embodiments, the method further comprises forming the first layer from an extruded polymeric film and forming a plurality of apertures in the extruded polymeric film. The apertures may be formed by mechanical embossing, stretch rupturing, vacuum forming, hydroforming, hydro-cutting, needle punching, ultrasonic, slitting, ring-rolling, and any combination thereof. The apertures may be formed before the bonding of first and second layers, simultaneously with the bonding of these layers, or afterwards, as a finishing step. Aperture-forming may be used to increase absorbency and extensibility of the absorbent material, if desired.

The bandage's absorbent pad is a thin absorbent bilayer that is processed without the use of high-pressure calendering, thereby substantially maintaining the three-dimensional structure and porosity of the materials. The individual shapes of these openings and uniformity across the whole pad layer of the bandage are precise and well maintained due to the pad's fiber and bilayer configuration, high precision cutting with ultrasonics or the like, and the absence of calendering pressure forces during manufacture.

In embodiments, the method further comprises thermally bonding the second layer to a third layer comprising an extruded polymeric apertured film. The second layer may be positioned between the first and third layers.

The method may comprise a second bonding step that includes thermal bonding, chemical bonding, ultrasonic bonding, embossing, and any combination thereof.

The second layer preferably comprises one or more thermally bondable fibers. The method may further comprise providing at least one non-thermally bondable fiber within the second layer. The non-thermally bondable fiber has a first melting point at least about 15° C. higher a second melting point of the thermally bondable fiber

In some embodiments, the method comprises arranging the thermally bondable fibers of the second layer in a core-sheath configuration, forming bicomponent fibers. In an exemplary embodiment, PET forms the core and polyethylene forms the sheath. The method may further comprise configuring the polyethylene sheaths randomly in the second layer, or in a substantially structured pattern of intersecting sheaths. Moreover, the method may comprise configuring them to be a single or multiple layers.

It is to be understood that both the foregoing general description and the following detailed description are exemplary and explanatory only and are not restrictive. Additional features will be set forth in part in the description which follows or may be learned by practice of the description.

### BRIEF DESCRIPTION OF THE DRAWINGS

The accompanying drawings, which are incorporated in and constitute a part of this specification, illustrate several embodiments of the disclosure and together with the description serve to explain the principles herein.

FIG. 1A is a perspective top view of a bandage having an adhesive layer and an absorbent pad;

FIG. 1B is a cross-sectional view of a bandage having an absorbent pad, an adhesive layer, and an additional layer;

FIG. 2 is a cross-sectional view of one embodiment of an absorbent pad; and

FIG. 3 is a perspective view of one embodiment of a bicomponent fiber of the absorbent pad of FIG. 2.

### DETAILED DESCRIPTION

This description and the accompanying drawings illustrate exemplary embodiments and should not be taken as limiting, with the claims defining the scope of the present description, including equivalents. Various mechanical, compositional, structural, and operational changes may be made without departing from the scope of this description and the claims, including equivalents. In some instances, well-known structures and techniques have not been shown or described in detail so as not to obscure the description. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

While the following is presented with respect to an absorbent pad for use with a bandage, it should be understood that the features described herein may be readily adapted for use in tending to wounds in various manners known to the art. For example, the absorbent article may be formed as a wrap-around dressing for the wound. It may be configured to contact the wound across its longitudinal surface and held in place by adhesives which do not extend across the surface of the pad opposite the skin. For example, only the outer boundaries of the pad may be held down onto the skin using adhesives or other techniques.

Moreover, it should be understood that the terms “absorbent pad” and “pad” as used in this specification and the appended claims do not restrict the purpose of the absorbent article to which they refer, to mere padding, serving simply as a cushioning or stuffing between other layers. In fact, the term may instead be directed broadly to a material which is thin, flat, and comprising fibers, such as the absorbent layer of a bandage known in the art.

The present description provides an absorbent pad and bandage for application to a wound. It should be understood that the absorbent pad and bandage are directed to treatment of any type of break or opening of epithelial tissue, such as skin. For example, a wound may be an abrasion, scraping, scab, blister, burn, incision, laceration, puncture, or an avulsion, and the absorbent material may be applied to the wound bed or the healing, closing tissue. The absorbent material may also be used on eschars, ulcers, and infected skin. Typical fluids which drain from a wound and enter the layer or layers of the absorbent pad include blood and its components therein, sweat, serous fluid, pus, and the like. The material may be used for wounds of any size, shape, or depth, and in a clinical setting or otherwise.

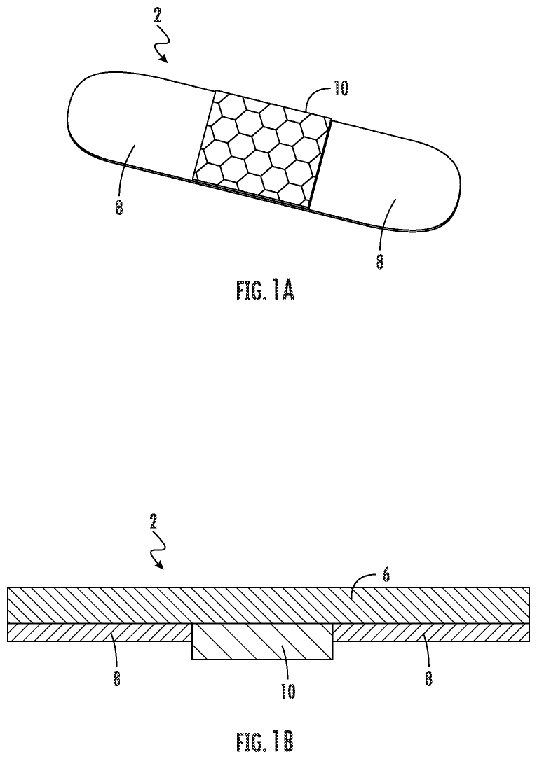

FIGS. 1A and 1B illustrate an illustrative bandage 2 having an adhesive layer 8 and an absorbent pad 10. An additional layer, such as a backing layer 6, may also be a layer of bandage 2. Bandage 2 may comprise one or more additional layers (not shown), or more than one layer of any shown in FIG. 1. In certain embodiments, a wound-release layer (not shown) may be added to the surface of absorbent pad 10 that contacts the wound. For example, a porous net may be placed between the wound and absorbent pad 10 to facilitate removal of the pad and/or bandage. It is understood that other types of backing or release layers may be added, and may include layers associated with easy removal of the pad or bandage from its packaging. Alternatively, a release layer may be present on the surface of adhesive layer 8 such as, for example, silicone paper release strips (not shown), which the user may remove before placement of the bandage onto skin.

In certain embodiments, adhesive layer 8 is placed on healthy tissue just outside the boundaries of the wound. Adhesive layer 8 may further be separated from the skin by another layer which acts as a skin barrier for those who have an allergy to particular adhesives, for example. In other embodiments of absorbent pad 10, allergies to adhesive material may also be avoided by using absorbent pad 10 as a wrap-around dressing over the wound instead of in bandage 2, as described previously. Adhesive layer 8 or backing layer 6 of bandage 2 may be any desired thickness. In some embodiments, backing layer 6 has a thickness in the range of about 1-4 mils. In other embodiments, each of these layers may be less than 1 mil thick.

In some embodiments, there may be another adhesive layer between absorbent pad 10 and backing layer 6. Alternatively, adhesives may be added directly to the surface of absorbent pad 10 which is adjacent to backing layer 6 before the two layers are combined in the forming of bandage 2.

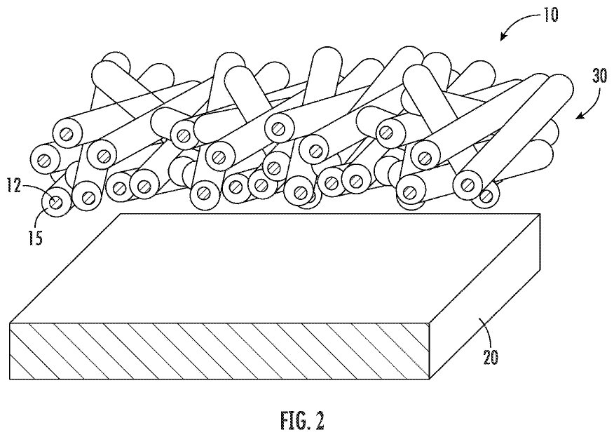

Referring now to FIG. 2, an embodiment of absorbent pad 10 is shown in cross-section with its first layer 20 and second layer 30 separated. Of course, it is understood that the separation of these layers shown in FIG. 2 serves only to clarify the features of absorbent pad 10 and not to imply that these layers are separated during use or remain separated as in the final product.

First layer 20 comprises a polymer and second layer 30 comprises at least one thermally bondable fiber. First and second layers 20, 30 are thermally bonded to each other to create a lightweight, and absorbent pad. Absorbent pad 10 has a thickness of about 40 mils to about 200 mils, preferably about 100 mils to about 130 mils. This range provides higher affinity bonding and reduces the amount of raw material required, thereby lowering higher manufacturing costs of the absorbent pad.

Absorbent pad 10 of bandage 2 exhibits high absorbency due to its unique structure. For example, absorbent pad 10 may have an absorbency between about 5 g/g and 100 g/g/or about 5 g/g to about 40 g/g, preferably about 10 g/g to about 20 g/g. In certain embodiments, the fibers are carded and subsequently air-through or thermally bonded to produce a web. In these embodiments, the fibers may have an absorbency of about 10 g/g to about 20 g/g, or about 14 g/g to about 16 g/g.

In other embodiments, the fibers may be spunbond and coated with a silicone-based coating (discussed below). The add-on weight of the silicone-based coating is about 1.0 gsm to about 20 gsm, or about 1.5 gsm to about 15.5 gsm. In an exemplary embodiment, the coated spunbond fibers have an absorbency of about 5 g/g to about 20 g/g, or about 10 g/g to about 12 g/g.

In embodiments, absorbent pad 10 comprises a third polymer layer (not shown). Second layer 30 may be positioned between the first and third layers. Second layer 30 may also be thermally bonded to the third layer. Absorbent pad 10 may include additional polymer layers and/or additional fiber layers.

First layer 20 and the third layer preferably comprises a polymeric film or mesh. Suitable films or meshes include apertured films, perforated films, polymeric meshes, microporous films, netting and the like. The meshes or films may include, non-woven materials, woven materials, knitted materials and the like.

In one embodiment, the polymeric film layers comprise an extruded apertured polymeric sheet or film. An apertured polymer film is a lightweight material that include apertures, pores, or perforations. The apertures may be embossed in a pattern (such as circular, diamond shaped, hexagonal, oblong, triangular, rectangular, etc.) and then stretched until apertures form in the thinned out areas created by the embossing. Such an apertured substrate can be formed from many polymers, such as polypropylene, polyethylene, high density polyethylene (“HDPE”) and the like.

Apertured films are used in many applications, including finger bandages, surgical gowns, drapes, masks, teeth whitening strips, hydrogel scrims, nasal support materials, electrode support products, filters, food processing, packaging and textile applications, agricultural products, food packaging, such as cheese production netting and many more. An apertured film is available commercially and is marketed by Schweitzer-Mauduit International, Inc. under the trademark Delnet®.

The apertures formed in first layer 20 and/or the third layer provide increased breathability, stretch and extensibility. The increase in flexibility is beneficial for ensuring that bandage 2 conforms to wounds which stretch open or contract when the user is in motion. Backing layer 6 may be impervious to fluids and other discharge from the wound so that these excretions may therefore be contained within or below absorbent pad 10. The apertures confer other important properties and will be described in greater detail below.

Persons skilled in the art will recognize that apertures 32 may be formed in other layers of bandage 2 besides the first and third layers. For example, apertures 32 may be formed in second layer 30, or they may extend through absorbent pad 10 and backing layer 6. In some embodiments in which bandage 2 comprises multiple layers additional to absorbent pad 10, apertures 32 differ in size and shape from one layer to the next. In other embodiments, bandage 2 and absorbent pad 10 need not comprise apertures 32 at all.

If apertures 32 are desired in only a single layer of pad 10, they may be introduced through first layer 20 (and/or third layer) before they are bonded with second layer 30. In some embodiments, apertures 32 may be formed through both layers before they are bonded together, or after, as a finishing step. In other embodiments, apertures 32 are introduced simultaneously with the bonding together of the pad's first and/or third layers with the second layer.

In some embodiments, apertures 32 may be shaped as polygons, such as, for example, hexagons, diamonds, triangles, octagons, squares, or rectangles. In other embodiments, apertures 32 may simply be linear or curvilinear slits through the material. In yet other embodiments, apertures 32 may be irregularly shaped or circular. They may range in size but preferably, are not so large that the general porosity of absorbent pad 10 is exceedingly high, as this will compromise the strength of pad 10. Moreover, apertures 32 may vary in size and/or shape across the surface of absorbent pad 10. The varied landscape of texture which results will create different wicking patterns across the surface of absorbent pad 10, should this be desired. One skilled in the art will recognize that the shape and size of apertures 32 will be decided on by determining the optimal porosity of regions across absorbent pad 10 as needed in a given application.

In certain embodiments, the apertures have a substantially uniform geometry to increase absorbency and extensibility of the absorbent article. They also serve to allow vapor and heat to escape the wound and provide the user with a textured surface that makes the absorbent article more comfortable, without causing a hot, moist, and plasticky feel on their skin.

In another embodiment, first layer 20 preferably comprises a polymeric mesh. In some embodiments, the polymeric mesh is made from polymeric resins and preferably, thermoplastic polymeric resins, which tend to confer strength to the pad. In preferred embodiments, the polymeric mesh is synthesized substantially from polyethylene articles. In other embodiments, other olefinic articles may be used, such as, for example, polypropylene, or blends of polyethylene and polypropylene. In yet other embodiments, the polymeric mesh additionally comprises other material such as polyester, rayon, cotton, or combinations thereof.

Second layer 30 comprises a plurality of thermally bondable fibers. In some embodiments, these fibers are bicomponent fibers having at least two different materials. The bicomponent fibers may be continuous (e.g., high loft) or discontinuous. The biocomponent fiber may comprise any suitable configuration, such as such as core/sheath with a concentric or eccentric core, side by side, segmented pie, island in the sea, hollow bicomponent fiber. hollow segmented pie, trilobal bicomponent fiber, mixed fibers, striped fibers, conductive fibers and the like. The bicomponent fiber may have a solid or a hollow core. For example, a segmented pie or side by side fiber may include a hollow core.

In embodiments, the bicomponent fiber comprises first and second polymer materials. The second polymer material has a lower melting temperature than the first polymer material. In an exemplary embodiment, the bicomponent fiber comprises a core and a sheath. The sheath comprises a material having a lower melting temperature than material of the core.

The core may comprise any suitable material, such as polyethylene terephthalate (PET), polylactic acid (PLA), polypropylene (PP) or combinations thereof. In an exemplary embodiment, the core comprises PET.

The sheath may comprise any suitable material, such as polyethylene (PE) high density polyethylene (HDPE), low-melting polyethylene terephthalate (CoPET), low-melting polylactic acid (PLA), polypropylene (PP) and combinations thereof. In an exemplary embodiment, the sheath comprises PE.

In one exemplary embodiment, the fibers in the second layer are arranged such that the PET forms a core surrounded by a PE sheath. The arrangement of the PET core and the PE sheath provide an easily bondable composition using thermal bonding due to, for example, the differences in melting temperature of the two components. Additionally, the method of thermally bonding the first and second layers to each other does not include high-pressure mechanical forces. Ultimately, these materials and their configuration and processing obviate the need for bonding techniques that disrupt the three-dimensional structure of the composite and allow the article to perform with greater efficiency.

In embodiments, the ratio by weight of the core and the sheath is about 30/70 to about 70/30, preferably about 40/60 to about 60/40. This range maintains optimal overall density of the absorbent pad. Density is an important consideration of the pad, as values too high or too low may render the pad uncomfortable or weak and ineffective. This range is also preferred because if the amount of core component is excessively high, thermal bonding at the melting point temperature of polyethylene will be poorer, and if it is excessively low, the material's tensile strength will be too low, resulting in an easily deformable thus a weak product

Although the preferred embodiment of a bicomponent fiber has a PET core surrounded by polyethylene, other polyester resins may be used as the core or the sheath. For example, PET can be replaced with polyethylene naphthalate, polylactic acid, and others. In some other embodiments, the configuration may be reversed such that the core comprises polyethylene that is surrounded by a sheath of PET. In other embodiments, polypropylene or other polymeric resins may form the core of the bicomponent fiber. Alternatively, the core and/or the sheath may be comprised of fibers which are blends of different raw materials.

In some embodiments, the fibers in the second layer comprise a spin finish. The spin finish is selected from hydrophilic or hydrophobic FDA approved (for skin contact) spin finishes. Preferably, it is selected from hydrophilic spin finishes so that liquid absorption capacity is increased. However, it should be recognized that the spin finish may be a non-FDA approved spin finish as long as it does not harm the skin. The spin finish is content is no more than 2%.

In embodiments, the second layer further comprises at least one non-thermally bondable fiber. The non-thermally bondable fiber has a first melting and the thermally bondable fiber has a second melting point wherein the first melting point is at least about 15° C. higher than the thermally bondable fiber. The non-thermally bondable fiber may comprise any suitable material, such as polyethylene terephthalate (PET), polybutylene terephthalate (PBT) polylactic acid (PLA), polypropylene (PP) or combinations thereof. For example, when the sheath is selected as HDPE, the non-bondable fiber could be PET.

The thermally bondable fiber may comprise a bicomponent fiber. The non-bondable fiber may have any suitable cross-section, such as round, non-round, 4DG, trilobal, ribbon or the like. Non-round fiber cross-sections have more specific surface area which results in more liquid absorption.

In embodiments, the non-thermally bondable fiber comprises 50% or less by weight of the second layer, preferably less than 40% or less by weight of the second layer.

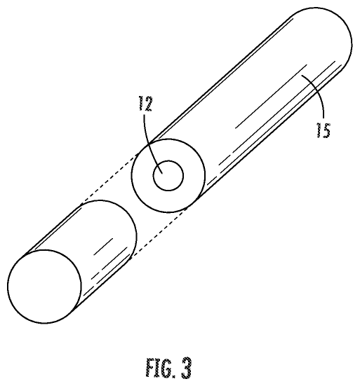

FIG. 3 illustrates a perspective view of one embodiment of a composite fiber of second layer 30 comprising a core-sheath configuration in which a PE sheath 15 surrounds a centrally located or concentric PET core 12. This core-sheath arrangement is beneficial for a number of reasons, particularly in allowing bonds to form between composite fibers without requiring mechanical pressing. Thermal bonding is sufficient to generate strong bonds between the fibers. For example, in some embodiments, thermal bonds can be achieved when the material comprising polyethylene sheath 15 with its PET core 12 is heated to a temperature at or above the temperature of the melting point of polyethylene sheath 15, which is a lower melting point than the melting point of PET core 12. In these embodiments, the polyethylene sheath 15 dissolves upon heating, it fuses with one or more nearby polyethylene sheaths while its more rigid PET core 12 maintains its structure to a higher degree than the sheath surrounding it. It is understood that this layer may be thermally bonded in other ways. For example, the temperature may be set to higher than the melting points of both, the core, and the sheath component, allowing the core fibers and the sheath fibers to melt and become bonded to each other.

Weak bonds between the different polymeric fibers and between layers of absorbent pad 10 result in easily deformable products. While calendering with very high pressures causes highly efficient fiber-to-fiber fusion, it can also cause melting of the material and introduces defects which result in poor performance during use. For example, high mechanical stress on the material achieved with calendering parameters commonly used in the art can compress the fibers such that they become flattened, openings are collapsed shut, and the material is effectively ‘clogged.’ This practice significantly reduces absorbency and can ultimately increase the risk of wound maceration when the final product is used.

In some embodiments, the core-sheath configuration of polyethylene sheath 15 with its PET core 12 is a centralized core-sheath configuration, in which PET core 12 is substantially located in the center of polyethylene sheath 15. This embodiment is illustrated in FIG. 3. Alternatively, these bi-composite fibers may be configured as eccentric core-sheaths, in which PET core 12 is positioned off-center (not shown). Eccentric core-sheath configurations may be desired if greater volume is needed in absorbent pad 10. When the core is off-center, a temperature set to the melting point of the sheath and below the melting point of the core fiber causes polyethylene sheath 15 and PET core 12 to shrink differently compared to one another and so the fiber begins to curl due to the differential shrinking taking placing off-center. This generates a “crimp” in the fiber and therefore, in the entire second layer 30, increasing volume within absorbent pad 10.

In other embodiments, the bicomponent fiber is engineered such that within each composite fiber, PET and polyethylene are located side by side when viewed in cross section, rather than in a core-sheath configuration. Those skilled in the art will recognize this configuration results in a highly crimped second layer 30. Alternatively, the bi-component fiber may comprise multiple PET fibers within each polyethylene sheath 15.

In one embodiment, a plurality of bi-composite fibers comprising polyethylene sheath 15 with a PET core 12 form second layer 30. In this embodiment, the plurality of polyethylene sheaths 15 are non-parallel to each other. Sheaths 15 may intersect each other in either the horizontal or vertical planes. Sheaths 15 may overlap with each other and form multiple layers. The sheaths 15 may each be confined to a single layer, or they may overlap across the layers.

In certain embodiments, the fibers are carded and subsequently air-through bonded to produce a web. In another embodiment, the fibers are spunbond by bonding together extruded spun filaments to create the web. The spunbond process may be more cost effective than other methods of producing the fibers and may create manufacturing efficiencies, such as higher throughput, lower maintenance, less space, increased automation, less scrap ratio, and a continuous manufacturing process (i.e., 24/7).

The silicone-based coating is applied by any suitable process including, but not limited to, spraying the fibers with the silicone-based coating, dipping the fibers in a vessel containing the silicone-based coasting, applying the silicone-based coating as a foam to the fibers, utilizing a metering rod or other leveling device to apply the coating, delivering the coating on the fibers with a coating head, such as a slot-die, or any combination of these techniques. In an exemplary embodiment, the coating is applied by spraying or dipping.

The silicone-based coating includes a silicone compound diluted in water or other suitable fluid such that the silicone compound comprises at least about 2 percent by weight of the coating, or at least about five percent by weight of the coating. In an exemplary embodiment, the silicone compound comprises about 10% by the weight of the coating. In an exemplary embodiment, the silicone compound comprises a silicone material, a surfactant, and water. The silicone and surfactant may together comprise about 5% to about 20%, or about 10% to about 11%, by weight of the overall coating.

In embodiments, the silicone-based coating comprises a reactive silicone macroemulsion. Silicone emulsions are insoluble silicones substantially evenly dispersed in water with the aid of a surfactant. The silicone emulsion may comprise, for example, dimethyl silicone emulsions, amino type silicone emulsions, organo-functional silicone emulsions, resin type silicone emulsions, film-forming silicone emulsions or the like. In an exemplary embodiment, the reactive silicone macroemulsion comprises an amino functional polydimethylsiloxane and/or a polyethylene glycol monotridecyl ether. In embodiments, the amino functional polydimethylsiloxane comprises about 30 to about 40 percent by weight of the coating. In embodiments, the polyethylene glycol monotridecyl ether comprises about 5 to about 10 percent by weight of the coating

In embodiments, the silicone-based coating further comprises an antistatic agent. The antistatic agent may comprise a surfactant. The surfactant may comprise a non-rewetting thermodegradable surfactant/foaming agent.

In various embodiments, the add-on weight of the silicone-based coating is about 1.0 gsm to about 20 gsm, or about 1.5 gsm to about 15.5 gsm. In certain embodiments, the add-on weight may be about 2.9 gsm to about 10.9 gsm.

Another desirable feature of absorbent pad 10 is its ability to separate the cell fraction of whole blood. Applicant has discovered that the absorbent material is capable of repelling cells from its center to its outer boundaries. This is due to the unique configuration of fibers in second layer 30. The ability to enrich for and isolate the cellular component onto a specific spatial region of a substrate would be advantageous in the clinical laboratory, for example, for performing nucleic acid extractions or quantification of low-abundance targets.

In one exemplary embodiment, a large bandage 2 with absorbent pad 10 may be administered onto a patient in a hospital with necrotized wound tissue. Given the severity of such infection and to prevent septicemia, the patient may be administered antibiotics as the wound heals. The ability to spatially separate the cellular component from fluid blood volume on or in absorbent pad 10 facilitates the collection and enrichment of bacterial cells from the wound. The portion of absorbent pad 10 containing the concentrated cells may be cut from the rest of the material and nucleic acid extractions may be performed solely on the cell-enriched portion in order to sequence and identify pathogens. In fact, cell enrichment is a major advantage in the filter-fabrics industry and in clinical settings where nucleic acid extractions are performed from patient blood samples, because blood is known to comprise major inhibitors of standard diagnostic techniques, like polymerase chain reaction (PCR).

Conversely, when it is desired to quantify serum biomarkers, the cell-enriched zone of pad 10 may be cut out and discarded. In these instances, the remaining fluid-filled portion containing the biomarkers of interest may be used for downstream processing.

Those skilled in the art will recognize that absorbent pad 10 may comprise additional materials that may be beneficial to bandage 2. For example, in certain embodiments, absorbent pad 10 may comprise antimicrobials which can be applied using a spin finish. Absorbent pad 10 may also comprise Benzalkonium chloride (BZK).

In some embodiments, bleed-control and/or other medical powders which are well known in the art may be added to absorbent pad 10. Hemostatic powders are commonly added to control bleeding. In some embodiments, a hemostatic powder is included in or on absorbent pad 10 for contacting the wound. The powder may contain chitosan salts, medical surfactants, and other treatments known in the art.

A method of making absorbent pad 10 for a wound dressing will now be provided. The method comprises the steps of providing a first polymer layer and a second layer comprising at least one thermally bondable fiber and thermally bonding or laminating the first layer to the second. In some embodiments, the thermally bondable fiber comprises a bicomponent fiber having first and second materials or components, such as those described above. The bicomponent fiber may be formed, for example, by selecting a heating temperature above the melting point of both components.

In certain embodiments, the bicomponent fiber is formed with a core/sheath configuration, as described above. The core/sheath fiber arrangement may be random or highly structured. The sheaths may be arranged in parallel, in a single or multilayer format. A layer comprising core-sheath conjugate fibers takes advantage of the different melting points of the core versus the sheath component. The difference in melting indices between the two components facilitates the bonding of the fibers using thermal bonding techniques. In some embodiments, the thermal bonding step allows the fusing of adjacent sheaths, sheaths with the core component, and/or sheaths with other resins if these are present. Absorbent pads may be processed to be a single layer of single- or multi-component fibers, or combinations of two or more layers of single-component or multi-component fibers, including layers of core-sheath configured fibers. When the appropriate temperatures are selected, thermal bonding may occur at the intersecting points between sheath fibers.

In an exemplary embodiment, PET and polyethylene are configured in a core-sheath arrangement. Though many different types of polymers may be used for either the core or the sheath, PET offers resiliency and loftiness to absorbent pad 10 and thus, is preferred as the core.

In other embodiments, the method comprises thermally bonding the first and second layers by selecting a heating temperature that renders the PET core 12 substantially unmelted, and polyethylene sheaths 15 are the fibers which are melted and thermally bonded to the first layer. In these embodiments, thermal bonding can occur at the melting point of the polyethylene sheath 15, which offers a lower melting point than that of PET core 12. With this approach, thermal bonding takes place substantially between just the sheaths and the first layer while maintaining a substantially semi-rigid or rigid PET core 12.

In embodiments, the method further comprises providing at least one non-thermally bondable fiber within the second or fiber layer. The non-thermally bondable fiber has a melting point that is at least about 15° C. higher than the melting point of the thermally bondable fiber. The non-thermally bondable fiber may comprise a bicomponent fiber. The non-bondable fiber may have any suitable cross-section, such as round, non-round, 4DG, trilobal, ribbon or the like. Non-round fiber cross-sections have more specific surface area which results in more liquid absorption. In embodiments, the non-thermally bondable fiber comprises 50% or less by weight of the second layer, preferably less than 40% or less by weight of the second layer.

In some embodiments, the method further comprises applying a spin finish to at least some of the fibers in the second layer. The spin finish is preferably no more than 2%.

In embodiments, the method further comprises forming the first layer from an extruded polymeric film and forming a plurality of apertures in the extruded polymeric film. The apertures may be formed by mechanical embossing, stretch rupturing, vacuum forming, hydroforming, hydro-cutting, needle punching, ultrasonic, slitting, ring-rolling, and any combination thereof. Ultrasonics cutting provides high precision in shaping the apertures and is thus preferred, however, any methods known in the art may be used.

The step of forming the aperture is important for enhancing absorbency and extensibility of the absorbent article. They provide an escape for water vapor and heat from the wound and provide a comfortable, cooled, and textured surface for the user. The step may be done to each layer separately or a single layer of the pad. If the apertures are desired in only a single layer of the pad, the aperture-forming step may be done to the first layer before first and second layers are bonded to each other. In some embodiments, the method comprises forming the apertures through both layers after the thermal bonding step of the bilayer, for example, as a finishing step. In yet other embodiments, the method comprises forming the apertures simultaneously with the bonding together of the bilayer.

The bandage's absorbent pad is a thin absorbent bilayer that is processed without the use of high-pressure calendering, thereby substantially maintaining the three-dimensional structure and porosity of the materials. The individual shapes of these openings and uniformity across the whole pad layer of the bandage are precise and well maintained due to the pad's fiber and bilayer configuration, high precision cutting with ultrasonics or the like, and the absence of calendering pressure forces during manufacture.

In embodiments, the method further comprises thermally bonding a third layer to the second layer such that the second layer is positioned between the first and third layers. Apertures may be formed in the third layer similar to the first layer described above. The apertures may be formed in the first and third layers simultaneously, or in separate steps.

In certain embodiments, the method may comprise a second bonding step. In preferred embodiments, the method comprises bonding the two layers using ultrasonic bonding. Alternatively, if thermal bonding is used for this step instead of ultrasonic bonding, the melting point temperature of polyethylene or polyethylene-blend may be applied. This allows the dissolving sheaths in the second layer 30 to then fuse with the polymer of the first layer with which it makes contact. The unique configuration disclosed herein allows one to avoid a step in its manufacturing which not only collapses pores and reduces the structural integrity of the absorbent pad but is also ubiquitously done in the art. While high-pressure calender rolling is eliminated in this method of making the pad, high-affinity bonding of the fibers and layers therein is still accomplished.

### Example 1

Applicant tested the water absorbency of the absorbent pads described herein. The absorbent pads were manufactured as described above. HDPE/PET bicomponent fibers ranging from 1.5 to 5 Denier were carded and subsequently air-through or thermally bonded. These nonwovens were then laminated onto an extruded apertured film with a minimal amount of pressure. The pads were cut into 3″×4″ samples and placed into a wire basket weighing about 7.5 grams with a 4″ tall cylinder and a 1″ radius. The container was filled with water deep enough to allow the basket to completely submerge. The samples were placed into the basket with the 4″ length extending in the machine direction (MD).

The wire basket was placed onto a plastic dish for weighing purposes. The samples were weighed to the nearest 0.01 gram (WS). The tare weight of the plastic dish (WG) and the test basket (WB) were also weighed to the nearest 0.01 gram. The sample was then placed into the basket with the 3″ edge parallel to the side of the basket. The basket was held with the sample closest to the water and dropped on its side from a height of 1″ into the container of water. The basket was allowed to remain submerged in the water for ten seconds. The basket and sample were then removed from the water to drain it for one minute.

Upon draining of the water, the basket and sample (Wt) were weighed in the plastic dish to the nearest 0.01 gram. The water absorptive capacity of the sample was then calculated in oz/sy based on the formula C=[Wt−(WB+WS+WG)]×3.82, wherein C=Capacity in oz/yd2, Wt=the total weight in grams of basket, plastic dish, sample and water after immersion, WB=the weight of the basket in grams, WG=the weight of the plastic dish in grams, WS=the weight of the sample, dry and before immersion in grams. 3.82 is the conversion factor.

Applicant tested three different sample absorbent pads as described above and calculated the average of these three test samples left, right and center as the value of liquid absorptive capacity. The capacity in oz/yd2 was then converted to g/g of the sample (i.e., net water gain weight (g)/initial material weight (g)). After multiple rounds of such testing, the values of the liquid absorption capacity of the samples were determined to be in the range of about 10 g/g to about 20 g/g.

### Example 2

Applicant tested the water absorbency of the absorbent pads described herein. HDPE/PET bicomponent continuous fibers ranging from 1.5 to 5 Denier were spunbond and then coated with a silicon-based coating as described above and then dried at 240° F. for 3 minutes. The silicone-based coating comprised a reactive silicone macroemulsion at 10% by weight, a non-rewetting thermodegradable surfactant/foaming agent at 1% by weight and water at 89% by weight. The bicomponent fibers had an eccentric sheath/core configuration. Samples were cut to a 3″×4″ size, laminated to an apertured film and then water absorption values were calculated.

Applicant tested two samples of spunbond HDPE/PET bicomponent fibers without the silicone-based coated (labeled “Uncoated Spunbond”) and two samples of spunbond HDPE/PET bicomponent fibers with the coating (labeled “Coated Spunbond”). Applicant further tested two samples of spunbond HDPE/PET bicomponent fibers that were carded and subsequently air-through bonded, as described in Example 1 (labeled “Carded”). The Carded samples did not include the silicone-based coating. The water absorption testing was conducted under the same standard described above in EXAMPLE 1. The result of this testing is shown below in TABLE 1.

TABLE 1

| | | Water | Water |
|---|---|---|---|
| | Basis Weight | Absorption | Absorption |
| Sample | (g/m²) | (g/g) | (oz/sy) |
| Uncoated Spunbond | 61.9 | 5.7 | 22 |
| Uncoated Spunbond | 72.9 | 2.6 | 10 |
| Coated Spunbond | 63.3 | 11.2 | 42.7 |
| Coated Spunbond | 69.3 | 11.3 | 43 |
| Carded | 87 | 15.8 | 60 |
| Carded | 86 | 14.9 | 57 |

As shown in TABLE 1, the silicone-based coating significantly improved the water absorption of the spunbond fibers (i.e., from a range of about 2.6 g/g to about 5.7 g/g in the uncoated samples to about 11.2 or 11.3 g/g in the coated samples). The carded samples had a higher water absorption than either the coated or uncoated spunbond samples. The carded samples also had a higher basis weight (about 86 or 87 g/m2).

Applicant further tested two samples of point bonded spunbond monocomponent PET fibers. The water absorption testing was conducted under the same standard described above in EXAMPLE 1. The first sample was uncoated and the second sample was coated with the silicon-based coating described above. The result of this testing is shown below in

TABLE 2

| | | Water | Water |
|---|---|---|---|
| | Basis Weight | Absorption | Absorption |
| Sample | (g/m²) | (g/g) | (oz/sy) |
| Uncoated | 90 | 2.6 | 10 |
| Monocomponent | | | |
| Coated | 97 | 3.48 | 13.3 |
| Monocomponent | | | |

As shown, the silicone-based coating improved the water absorption of the monocomponent fibers. However, the overall water absorption for both the coated and uncoated samples was significantly less than the biocomponent Carded samples and Coated Spunbond samples shown in TABLE 1. Thus, the bicomponent configuration described herein significantly increases the water absorption of the fibers.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

For example, in a first aspect, a first embodiment is an absorbent pad for a wound dressing comprising a first layer comprising a polymer and a second layer thermally bonded to the first layer and comprising at least one thermally bondable fiber.

A second embodiment is the first embodiment, wherein the first layer comprises a polymeric film.

A third embodiment is any combination of the first two embodiments, wherein the first layer comprises an extruded film having apertures.

A 4th embodiment is any combination of the first 3 embodiments, wherein the apertures comprise pores or perforations.

A 5th embodiment is any combination of the first 4 embodiments, wherein the apertures are hexagonal, square, diamond shaped. circular, oblong, triangular, rectangular or combinations thereof.

A 6th embodiment is any combination of the first 5 embodiments, wherein the first layer comprises an apertured film, a perforated film, a microporous film, a netting, a polymeric mesh, a knitted material, or combinations thereof.

A 7th embodiment is any combination of the first 6 embodiments, further comprising a third layer comprising a polymer.

An 8th embodiment is any combination of the first 7 embodiments, wherein the second layer is positioned between the first and third layers.

A 9th embodiment is any combination of the first 8 embodiments, wherein the third layer comprises a polymeric film.

A 10th embodiment is any combination of the first 9 embodiments, wherein the third layer comprises an extruded apertured film.

An 11th embodiment is any combination of the first 10 embodiments, wherein the thermally bondable fiber comprises a bicomponent fiber.

A 12th embodiment is any combination of the first 11 embodiments, wherein the biocomponent fiber comprises a core and a sheath.

A 13th embodiment is any combination of the first 12 embodiments, wherein the core comprises a first material and the sheath comprises a second material, wherein the second material has a lower melting temperature than the first material.

A 14th embodiment is any combination of the first 13 embodiments, wherein the core comprises a material selected from the group consisting of polyethylene terephthalate (PET), polylactic acid (PLA), polypropylene (PP) or combinations thereof.

A 15th embodiment is any combination of the first 14 embodiments, wherein the sheath comprises a material selected from the group consisting of polyethylene (PE) high density polyethylene (HDPE), low-melting polyethylene terephthalate (CoPET), low-melting polylactic acid (PLA), polypropylene (PP) and combinations thereof.

A 16th embodiment is any combination of the first 15 embodiments, wherein a ratio by weight of the core and the sheath is about 30/70 to about 70/30.

A 17th embodiment is any combination of the first 16 embodiments, wherein the ratio is about 40/60 to about 60/40.

An 18th embodiment is any combination of the first 17 embodiments, wherein the bicomponent fiber is selected from a group consisting of side by side, segmented pie, island in the sea, hollow bicomponent fiber. hollow segmented pie, trilobal bicomponent fiber, mixed fibers, striped fibers, conductive fibers, and combinations thereof.

A 19th embodiment is any combination of the first 18 embodiments, wherein the bicomponent fiber comprises first and second polymer materials, wherein the second polymer material has a lower melting temperature than the first polymer material.

A 20th embodiment is any combination of the first 19 embodiments, wherein a ratio by weight of the first and second polymer materials is about 20/80 to about 80/20.

A 21st embodiment is any combination of the first 20 embodiments, wherein the second layer comprises at least one non-thermally bondable fiber.

A 22nd embodiment is any combination of the first 21 embodiments, wherein the non-thermally bondable fiber comprises a bicomponent fiber.

A 23rd embodiment is any combination of the first 3 embodiments, wherein the non-thermally bondable fiber has a first melting and the thermally bondable fiber has a second melting point, wherein the first melting point is at least about 15° C. higher than the thermally bondable fiber.

A 24th embodiment is any combination of the first 23 embodiments, wherein the non-thermally bondable fiber comprises a material selected from the group consisting of polyethylene terephthalate (PET), polybutylene terephthalate (PBT) polylactic acid (PLA), polypropylene (PP) or combinations thereof.

A 25th embodiment is any combination of the first 24 embodiments, wherein the non-thermally bondable fiber comprises 50% or less by weight of the second layer.

A 26th embodiment is any combination of the first 25 embodiments, wherein the non-thermally bondable fiber comprises less than 40% or less by weight of the second layer.

A 27th embodiment is any combination of the first 26 embodiments, wherein the absorbent pad has a thickness in the range of about 40 mils to about 200 mils.

A 28th embodiment is any combination of the first 27 embodiments, wherein the absorbent pad has a liquid absorption capacity of about 5 g/g to about 100 g/g.

A 29th embodiment is any combination of the first 28 embodiments, wherein the absorbent pad has a liquid absorption capacity of about 10 g/g to about 20 g/g.

A 30th embodiment is any combination of the first 29 embodiments, wherein the thermally bondable fiber is formed from carding and has a liquid absorption capacity of at least about 14 g/g.

A 31st embodiment is any combination of the first 30 embodiments, wherein the thermally bondable fiber is spunbond.

A 32nd embodiment is any combination of the first 31 embodiments, wherein the thermally bondable fiber is coated with a silicone compound.

A 33rd embodiment is any combination of the first 32 embodiments, wherein the silicone compound is between about 1.5% and about 15% by weight of a total weight of the second layer.

A 34th embodiment is any combination of the first 33 embodiments, wherein the thermally bondable fiber has a liquid absorption capacity of at least about 10 g/g.

A 35th embodiment is any combination of the first 34 embodiments, wherein the second layer has a basis weight of about 60 gsm to about 75 gsm.

A 36th embodiment is any combination of the first 35 embodiments, wherein the silicone-based coating comprises a reactive silicone macroemulsion.

In another aspect, a bandage is provided comprising the absorbent pad of any combination of the first 36 embodiments.

In another aspect, a wound dressing is provided comprising the absorbent pad of any combination of the first 36 embodiments.

In another aspect, a first embodiment is a bandage comprising an adhesive layer and an absorbent pad bonded to and extending across at least a portion of the adhesive layer. The absorbent pad comprises a first polymer layer and a second layer thermally bonded to the first layer and comprising at least one thermally bondable fiber.

A second embodiment is the first embodiment, wherein the first layer comprises an extruded polymeric film having apertures.

A 3rd embodiment is any combination of the first 2 embodiments, further comprising a third layer comprising an extruded polymeric apertured film, wherein the second layer is positioned between the first and third layers.

A 4th embodiment is any combination of the first 3 embodiments, wherein the thermally bondable fiber comprises a biocomponent fiber.

A 5th embodiment is any combination of the first 4 embodiments, wherein the biocomponent fiber comprises a core and a sheath.

A 6th embodiment is any combination of the first 5 embodiments, wherein the core comprises a first material and the sheath comprises a second material, wherein the second material has a lower melting temperature than the first material.

A 7th embodiment is any combination of the first 6 embodiments, wherein the core comprises a material selected from the group consisting of polyethylene terephthalate (PET), polylactic acid (PLA), polypropylene (PP) or combinations thereof.

An 8th embodiment is any combination of the first 7 embodiments, wherein the sheath comprises a material selected from the group consisting of polyethylene (PE) high density polyethylene (HDPE), low-melting polyethylene terephthalate (CoPET), low-melting polylactic acid (PLA), polypropylene (PP) and combinations thereof.

A 9th embodiment is any combination of the first 8 embodiments, wherein a ratio by weight of the core and the sheath is about 30/70 to about 70/30.

A 10th embodiment is any combination of the first 9 embodiments, wherein the bicomponent fiber is selected from a group consisting of side by side, segmented pie, island in the sea, hollow bicomponent fiber. hollow segmented pie, trilobal bicomponent fiber, mixed fibers, striped fibers, conductive fibers, and combinations thereof.

An 11th embodiment is any combination of the first 10 embodiments, wherein the second layer comprises at least one non-thermally bondable fiber having a first melting point and the thermally bondable fiber has a second melting point wherein the first melting point is at least about 15° C. higher than the thermally bondable fiber.

A 12th embodiment is any combination of the first 11 embodiments, wherein the non-thermally bondable fiber comprises a material selected from the group consisting of polyethylene terephthalate (PET), polybutylene terephthalate (PBT) polylactic acid (PLA), polypropylene (PP) or combinations thereof.

A 13th embodiment is any combination of the first 12 embodiments, wherein the absorbent pad has a thickness in the range of about 40 mils to about 200 mils.

A 14th embodiment is any combination of the first 13 embodiments, wherein the absorbent pad has an absorbency of about 5 g/g to about 100 g/g.

A 15th embodiment is any combination of the first 14 embodiments, wherein the second layer has a liquid absorption capacity of about 10 g/g to about 20 g/g.

A 16th embodiment is any combination of the first 15 embodiments, wherein the thermally bondable fiber is formed from carding and has a liquid absorption capacity of at least about 14 g/g.

A 17th embodiment is any combination of the first 16 embodiments, wherein the thermally bondable fiber is spunbond.

An 18th embodiment is any combination of the first 17 embodiments, wherein the thermally bondable fiber is coated with a silicone-based coating comprising a silicone compound.

A 19th embodiment is any combination of the first 18 embodiments, wherein the thermally bondable fiber has a liquid absorption capacity of at least about 10 g/g.

In another aspect, a first embodiment is method of making an absorbent pad for a wound dressing or a bandage. The method comprises providing a first polymer layer and a second layer comprising at least one thermally bondable fiber and thermally bonding the first layer to the second layer.

A second embodiment is the first embodiment, further comprising forming a plurality of apertures in the first layer.

A 3rd embodiment is any combination of the first 2 embodiments, wherein the plurality of apertures is formed by one of mechanical embossing, stretch rupturing, vacuum forming, hydroforming, hydro-cutting, needle punching, ultrasonic, slitting, ring-rolling, and any combination thereof.

A 4th embodiment is any combination of the first 3 embodiments, further comprising forming the first layer from an extruded polymeric film and forming a plurality of apertures in the extruded polymeric film.

A 5th embodiment is any combination of the first 4 embodiments, further comprising thermally bonding the second layer to a third layer comprising an extruded polymeric apertured film, wherein the second layer is positioned between the first and third layers.

A 6th embodiment is any combination of the first 5 embodiments, wherein the thermally bondable fiber comprises a biocomponent fiber, the method further comprising forming a sheath around one or more core fibers.

A 7th embodiment is any combination of the first 6 embodiments, wherein the core fibers comprise a material selected from the group consisting of polyethylene terephthalate (PET), polylactic acid (PLA), polypropylene (PP) or combinations thereof.

An 8th embodiment is any combination of the first 7 embodiments, wherein the sheath comprises a material selected from the group consisting of polyethylene (PE) high density polyethylene (HDPE), low-melting polyethylene terephthalate (CoPET), low-melting polylactic acid (PLA), polypropylene (PP) and combinations thereof.

A 9th embodiment is any combination of the first 8 embodiments, further comprising providing at least one non-thermally bondable fiber within the second layer, the non-thermally bondable fiber having a first melting point and the thermally bondable fiber having a second melting point, wherein the first melting point is at least about 15° C. higher than the thermally bondable fiber.

A 10th embodiment is any combination of the first 9 embodiments, wherein the non-thermally bondable fiber comprises a material selected from the group consisting of polyethylene terephthalate (PET), polybutylene terephthalate (PBT) polylactic acid (PLA), polypropylene (PP) or combinations thereof.

An 11th embodiment is any combination of the first 10 embodiments, further comprising forming the second layer by carding a plurality of thermally bondable fibers.

A 12th embodiment is any combination of the first 11 embodiments, further comprising forming the second layer by spun bonding a plurality of thermally bondable fibers.

A 13th embodiment is any combination of the first 12 embodiments, further comprising coating the plurality of thermally bondable fibers with a silicon compound.

In another aspect, an absorbent pad is provided that is made from the process of any combination of the above 13 embodiments.

In another aspect, a bandage is provided that is made from the process of any combination of the above 13 embodiments.

What is claimed is:

1. An absorbent pad for a wound dressing, the absorbent pad comprising:
a first layer comprising a polymer; and
a second layer thermally bonded to the first layer and comprising at least one thermally bondable fiber.

2. The absorbent pad of claim 1, wherein the first layer comprises an extruded film having apertures.

3. The absorbent pad of claim 1, wherein the thermally bondable fiber comprises a bicomponent fiber.

4. The absorbent pad of claim 3, wherein the biocomponent fiber comprises a core and a sheath.

5. The absorbent pad of claim 4, wherein the core comprises a first material and the sheath comprises a second material, wherein the second material has a lower melting temperature than the first material.

6. The absorbent pad of claim 5, wherein the core comprises a material selected from the group consisting of polyethylene terephthalate (PET), polylactic acid (PLA), polypropylene (PP) or combinations thereof.

7. The absorbent pad of claim 5, wherein the sheath comprises a material selected from the group consisting of polyethylene (PE) high density polyethylene (HDPE), low-melting polyethylene terephthalate (CoPET), low-melting polylactic acid (PLA), polypropylene (PP) and combinations thereof.

8. The absorbent pad of claim 5, wherein a ratio by weight of the core and the sheath is about 30/70 to about 70/30.

9. The absorbent pad of claim 3, wherein the bicomponent fiber comprises first and second polymer materials, wherein the second polymer material has a lower melting temperature than the first polymer material.

10. The absorbent pad of claim 9, wherein a ratio by weight of the first and second polymer materials is about 20/80 to about 80/20.

11. The absorbent pad of claim 1, wherein the second layer comprises at least one non-thermally bondable fiber.

12. The absorbent pad of claim 11, wherein the non-thermally bondable fiber comprises a bicomponent fiber.

13. The absorbent pad of claim 12, wherein the non-thermally bondable fiber has a first melting and the thermally bondable fiber has a second melting point, wherein the first melting point is at least about 15° C. higher than the thermally bondable fiber.

14. The absorbent pad of claim 11, wherein the non-thermally bondable fiber comprises a material selected from the group consisting of polyethylene terephthalate (PET), polybutylene terephthalate (PBT) polylactic acid (PLA), polypropylene (PP) or combinations thereof.

15. The absorbent pad of claim 11, wherein the non-thermally bondable fiber comprises 50% or less by weight of the second layer.

16. The absorbent pad of claim 1, wherein the absorbent pad has a thickness in the range of about 40 mils to about 200 mils.

17. The absorbent pad of claim 1, wherein the absorbent pad has a liquid absorption capacity of about 5 g/g to about 100 g/g.

18. The absorbent pad of claim 1, wherein the absorbent pad has a liquid absorption capacity of about 10 g/g to about 20 g/g.

19. The absorbent pad of claim 1, wherein the thermally bondable fiber is formed from carding and has a liquid absorption capacity of at least about 14 g/g.

20. The absorbent pad of claim 1, wherein the thermally bondable fiber is spunbond.

21. The absorbent pad of claim 20, wherein the thermally bondable fiber is coated with a silicone compound.

22. The absorbent pad of claim 21, wherein the silicone compound is between about 1.5% and about 15% by weight of a total weight of the second layer.

23. The absorbent pad of claim 21, wherein the thermally bondable fiber has a liquid absorption capacity of at least about 10 g/g.

24. The absorbent pad of claim 21, wherein the second layer has a basis weight of about 60 gsm to about 75 gsm.

25. The absorbent pad of claim 21, wherein the silicone-based coating comprises a reactive silicone macroemulsion.

26. A wound dressing comprising the absorbent pad of claim 1.
